(19) United States
(12) Patent Application Publication
(10) Pub. No.: US 2023/0292647 A1
(43) Pub. Date: Sep. 21, 2023

(54) System and Method for Crop Monitoring

(71) Applicant: Dark Horse Technologies Ltd, Covent Garden (GB)

(72) Inventors: Jared Bainbridge, Kensington (GB); Grant Whittle, Houston, TX (US)

(21) Appl. No.: 18/010,895

(22) PCT Filed: Jun. 16, 2021
(86) PCT No.: PCT/GB2021/051535

§ 371 Date: Dec. 16, 2022

(30) Foreign Application Priority Data
Jun. 16, 2020 (GB) 2009146.8
Feb. 1, 2021 (GB) 2101329.7

(51) Int. Cl. A01B 79/00; A01B 79/02; G06T 7/00; G06V 20/10; G06V 20/17; G06V 10/82

(52) U.S. Cl. CPC A01B 79/005; A01B 79/02; G06T 7/0016; G06V 20/188; G06V 20/17; G06V 10/82; G06T 2207/10028; G06T 2207/10036; G06T 2207/20081; G06T 2207/20084; G06T 2207/30188

(57) ABSTRACT

Disclosed is a method of automated crop monitoring based on the processing and analysis of a large number of high resolution aerial images that map an area of interest using computer vision and machine learning techniques. The method comprises receiving 120 or retrieving image data containing a plurality of high resolution images of crops in an area of interest for monitoring, identifying 130 one or more crop features of each crop in each image, determining 140, for each identified crop feature, one or more crop feature attributes, and generating or determining 160 one or more crop monitoring outputs based, at least in part, on the crop features and crop feature attributes. Also disclosed is a method generating field camera specific training data for the machine learning model used to analyse the received image data.

### TECHNICAL FIELD

This invention relates generally to a method and system of automated crop monitoring based on analysing a plurality of images of crops in an area of interest. The invention also relates to a method of generating training data for the method and system.

### BACKGROUND TO THE INVENTION

Agriculture faces many challenges. Farmers typically pre-sell their crop. The efficiency of food production or the crop yield, i.e. the amount of crop harvested per unit area of land (e.g. kilograms/hectare or metric tons/hectare), is therefore an important metric to estimate, predict and maximise as it directly impacts the revenue of the farmer and the wholesale value of the crop produce. It is also important to predict food security.

At present, crop yield is predicted using traditional methods involving manually sampling small areas of the field and extrapolating to the total area of the field, or using modern remote sensing techniques such as correlating normalised difference vegetation index (NDVI) obtained from satellite imaging of the field with expected yield at harvest. However, despite NDVI being an extremely useful and rapid crop monitoring tool, both methodologies are inherently inaccurate, producing yield predictions with up to a +/−33% margin of error. This high margin of error results in low confidence in the final yield at harvest, often forcing farmers to under-sell their crop in order to help ensure they can meet their contractual obligations.

In addition, farmers lose on average 30-35% of crop produce (such as cereals, soybean, cotton and sugarcane) before it is harvested due to disease, weeds, pests. Farmers spend substantial sums protecting their crops from these crop-loss events. Crop growth/health/condition typically varies throughout a field based a range of factors such as the presence of microclimates, different parts of the field receiving more or less nutrients, water, or sunlight, crop-loss events (diseases, pests and weeds), or the soil being more or less fertile. Farmers currently spend many hundreds of hours per year walking, surveying or driving over their land to manually inspect the crops and look for signs of problems. Once a problem is identified, corrective actions can be implemented, such as watering, fertilising, treating and otherwise intervening in crops.

However, aside from this manual process being extremely time consuming, it is heavily reliant on the knowledge and experience of the individual to spot and diagnose a problem. It also relies on the farmer actually passing a problem crop or area to see it. Once a problem is found (e.g. stripe rust in wheat), the corrective action/intervention (e.g. fungicide) is typically applied to the whole field because the farmer is unable to quantify how much of the field is actually effected, potentially wasting valuable resources and increasing the production cost of the crop. This process is especially inaccurate and inefficient for large areas of land. Accurate yield prediction and crop-loss mitigation are both a focus for farmers and at the moment there are no remote-sensing solutions which are able to diagnose and map crop-loss events at scale.

There is therefore a need for an automated system and method of crop monitoring for more efficient crop monitoring, accurate crop yield predictions, and highly targeted interventions up to a plant-by-plant level.

Aspects and embodiments of the present invention have been devised with the foregoing in mind.

### SUMMARY OF THE INVENTION

According to a first aspect of the invention, there is provided a method of automated crop monitoring. The method comprises receiving or retrieving image data containing a plurality of (high resolution) images of crops in an area of interest for monitoring. The method may also comprise identifying one or more crop features of each crop in each image. The method may also comprise determining, for each identified crop feature, one or more crop feature attributes. The method may further comprise generating or determining one or more crop monitoring outputs based, at least in part, on the crop features and crop feature attributes. The one or more crop feature attributes may include any one or more of: location, colour, dimension/size, sub-feature count, diseased/disease type, pest-ridden/pest type, weed-ridden/weed type, healthy/unhealthy, normalised difference vegetation index (NDVI), normalised difference water index (NDWI), surface soil moisture (SSM) index, soil organic carbon (SOC) level/index, bare soil index (BSI), visual atmospheric resistance index (VARI), a digital elevation model (DEM).

In this context a crop means a single plant, such as wheat, barley, maize (corn), etc. A crop feature means a feature of interest of the crop, such as a head, stem or leaf, which may have sub-features such as kernels (grains) on a head. An area of interest (AOI) may be a field or a region of a field containing the crops.

The method provides for autonomous identification and analysis of multiple crop features and attributes that can be used to define a crop's overall health, yield, and dimensions. The method can provide accurate yield predictions, crop-loss diagnosis and intervention monitoring. The method is based on the processing and analysis of a large number of high resolution aerial images that map the AOI using computer vision and machine learning techniques. The method of autonomous monitoring is significantly more efficient, reliable and detailed/complete than conventional manual monitoring methods. It removes the reliance on knowledge and experience of individual farmers or farm workers and the element of chance involved in manually inspecting crops in an AOI. In the present invention, every crop in the AOI captured and resolved in the images is automatically analysed to identify each crop feature to determine its attributes in a rapid and consistent manner across the whole AOI.

The images are high resolution (HR) digital aerial images of the area of interest (AOI). The images may be image frames/still images. The image data may comprise video data from which the plurality of images are extracted as image frames/still images. The images may be referred to as “field” images generated by a “field” camera. The field camera may be or comprise a high resolution multi-spectral camera.

Each image captures a small region of the AOI with a sufficiently high resolution in order to resolve individual crops and crop features in the AOL. The required pixel resolution (pixels per meter) of the images is dependent on the type and size of crop being monitored, but preferably the image has a pixel resolution of at least 32 pixels per meter, and preferably greater than 128 or 256 pixels per meter, and/or a pixel size of less than 25 mm, and preferably less than 10, 5 or 1 mm. Each image may be a HR image of a small sub-region of the AOL.

Each image may be a multispectral image with a plurality of spectral bands or channels. Each spectral band or channel may have an associated spectral response characterising the sensitivity of the spectral band or channel to light of different wavelengths. Each image may have an image resolution. The image resolution may be defined by a pixel resolution, spatial resolution and/or spectral resolution. Each image comprises a plurality of pixels, each with a pixel value in one or more different wavelength/spectral bands or channels, as well as a geographic location and a date/time stamp. Geo-location and date/time stamp may be held as metadata in the respective image. As such, the images contain the necessary geo-location information (i.e. longitude and latitude) to reference them to a particular crop and to each other.

Each image may be mapped to a different geolocation in the AOI (e.g. using the geographic location information). The images may comprise an array of overlapping images, where each image is mapped to a different geolocation in the AOI. The plurality of images may include multiple viewpoints of each crop in the AOI. For example, each crop may appear in multiple images, where each image contains a different viewpoint of the crop. Adjacent images may overlap by up to 95% in each direction, e.g. X and Y directions, such that the same crop is captured in multiple images and each image contains a different viewpoint of the crop.

The images may be orthoimages and/or form or be used to form an orthomosaic map of the AOI. Where the images form an orthomosaic map, this may be generated by stitching a plurality of overlapping HR drone images.

An orthoimage is an aerial image that has been geometrically corrected (“orthorectified”) such that the scale is spatially uniform across the image. As such, an orthoimage has the same lack of distortion as a conventional map. Unlike an uncorrected aerial image, an orthoimage can be used to measure true distances, because it is an accurate representation of the Earth's surface, having been adjusted for topographic relief, lens distortion, and camera tilt. Images can be orthorectified using well known image processing techniques. An orthomosaic map is a single composite orthoimage which is an accurate visual representation of an area generated from many individual smaller images that have been stitched together. The individual images must overlap with each other to create the orthomosaic map. Orthomosaic maps can be generated well known image processing techniques. Generating an orthomosaic map may comprise obtaining an array of overlapping images and stitching the array of images to form the orthomosaic map.

The images may be colour images, whereby each pixel comprises a pixel value in a plurality of colour channels/bands, such as red, green, and blue (RGB) (or equivalently, hue, saturation and value (HSV)). The images may additionally comprise one or more infrared (IR) bands/channels, e.g. near-IR (NIR, 700-1100 nm) and/or shortwave IR (SWIR, 1100-3000 nm). For example, satellite images and many modern digital cameras can have RGB and IR channels. Alternatively, the image data may comprise separate IR images.

The HR images can be taken or generated by a camera mounted to an unmanned aerial vehicle (UAV) (i.e. a “drone” images) or by a satellite (satellite images), or manually captured images via a camera or smart phone. Drones are a convenient delivery mechanism for capturing the spatial resolution necessary for the method, but are not essential. Where used, drone images may be captured/generated at a constant altitude/height above ground level (AGL) in a drone survey. The altitude AGL for generating the HR images may be in the range 5 m to 50 m, and preferably 10-20 m. Lower resolution images may be obtained from greater altitudes AGL, e.g. >100 m.

The one or more crop monitoring outputs may include one or more of: a crop feature population count, a crop feature population density map, a volumetric crop yield prediction, a crop loss map, a diseased crop map, and/or one or more intervention instructions.

The one or more intervention instructions may comprise instructions to apply one or more treatments to one or more regions of the AOI. Optionally or preferably, the instructions may be machine integrated instructions for one or more agricultural machinery units or vehicles to apply the one or more treatments to the one or more regions. The one or more treatments may include any one or more of: applying water, seeds, nutrients, fertiliser, fungicide (e.g. for disease treatment), herbicide (e.g. for weed killing), and any other disease and/or weed killing treatments or mitigation interventions.

The one or more interventions may include identifying high-productivity regions in an AOI, identifying regions for targeted seed planting, targeted fertilising (nitrogen, phosphorus, potassium) and other yield improvement measures, herbicides (e.g. targeted black grass spray interventions) and other weed killing interventions, targeted fungicides and other disease treatments and mitigation interventions, water stress/pooling monitoring with targeted interventions; as well as monitoring the effectiveness of any of these interventions over time. Intervention techniques may include nitrogen applications, pesticides, irrigation.

The method therefore generates practical quantitative data and outputs that a farmer can use to maintain the crops, predict yield, diagnose crop loss. Farmers can use the output of the method to take proactive intervention steps to protect yield through early detection and diagnosis of crop loss events such as disease, pests and weeds.

In an embodiment, the crop features are identified, and certain (secondary) crop feature attributes are determined using computer vision and machine learning techniques applied to the images. By using a machine learning model trained using input from an experienced farmer or farm worker, all the crops in the AOI can be analysed in a consistent manner using the same expert insight. This approach leverages expert knowledge and takes away the person to person variability in traditional methods involving manual crop inspections.

Identifying crop features may comprise detecting and classifying one or more crop features in each image. The one or more crop features in each image may be identified using a machine learning model trained on a dataset of crop images to identify the one or more crop features in the respective image based, at least in part, on one or more image features extracted from each respective image. Optionally or preferably, identifying a crop feature includes identifying a crop feature type (e.g. head, stem, leaf, etc.).

The crop feature attributes may comprise primary crop feature attributes that are derivable directly from the images, i.e. image pixel attributes/values and/or structural features in the image. The crop feature attributes may additionally or alternatively comprise secondary crop feature attributes that are derivable/determined indirectly from the images using computer vision and machine learning techniques which involve making predictions based on the image data. The crop feature attributes may further comprise tertiary crop feature attributes that are derivable using additional input data, such as additional image data (e.g. satellite imagery of the AOI), weather data, and/or ground control data (from user input). The additional input data may be external to the received image data. Where the additional input data comprises additional image data, this may be contained in the received image data. Weather data may be used to determine if the crop feature has collected enough rain or too much rain, and/or enough sun (UV) or not enough sun, and/or the temperature conditions. User input(s) may include historical yield data, seed date, and/or sow date.

Determining the one or more crop features attributes may comprise extracting one or more primary crop feature attributes from each identified crop feature based on the image pixel values and/or based on one or more image features extracted from each respective image. The one or more primary crop feature attributes may include any one or more of: a location, a colour, a dimension, a normalised difference vegetation index (NDVI), and a sub-feature count. The location of each crop feature may be determined, at least in part, using geolocation data/information of each respective image in the image data.

The primary attributes may be based on pixels or pixel attributes of the respective identified crop feature. Pixel attributes may include the pixel size, location and value in each spectral band. Pixel attributes of an identified crop feature may include a collective attribute of the pixels in the region(s) identified as a crop feature, such as a dimension (width, length), aspect ratio, principle axis, area, average pixel value (e.g. dominant colour), or other statistical property derived from the pixels making up the identified crop feature.

The one or more primary crop feature attributes may comprise one or more geometric and/or spectral attributes derived from the pixels or pixel attributes of the respective identified crop feature. Geometric attributes may be derived from the size and location of pixels in the identified crop feature. The geometric attributes may include one or more of: location, dimension, area, aspect ratio, sub-feature size and/or count. Spectral attributes may be derived from the pixel values (intensities) in one or more spectral bands of pixels of the identified crop feature. The spectral attributes may include one or more of: dominant colour, RGB, red edge and/or NIR pattern, normalised difference vegetation index (NDVI), and normalised difference water index (NDWI). A pattern may be a collection of features such as edges or blobs.

Determining the one or more crop features attributes may comprise determining, or detecting and classifying, one or more secondary crop feature attributes for each identified crop feature using a machine learning model trained on a dataset of crop images to determine the one or more secondary crop feature attributes based, at least in part, on one or more image features extracted from each respective image and/or based at least in part on the determined primary crop features attributes. The one or more secondary crop feature attributes may include one or more of: diseased and disease type, pest-ridden and pest type, weed-ridden and weed type, healthy, and unhealthy. The primary crop features attributes may be used and be helpful for detecting secondary crop feature attributes. For example, black grass (a secondary attribute) is clearly visible once a kernel can be detected on a wheat plant. So detecting the kernel (primary attribute) helps to identify the secondary attribute (i.e. a disease, or if the kernel is healthy, unhealthy).

For example, the machine learning model may be trained to detect: various diseases such as septoria, stripe rust, leaf rust and fusarium head blight (FHB); various pests such as aphids, mites, orange wheat blossom midge (OWBM), locusts; and/or various weeds such as black grass. A crop feature may be classed as healthy if no disease, pests or weeds are detected. Crop feature “health” may also be deduced from its size/dimensions compared to the expected size/dimensions for the current crop cycle.

Determining the one or more crop features attributes may comprise determining one or more tertiary crop feature attributes for each identified crop feature based on additional or external input data. The one or more tertiary crop feature attributes may include any one or more of: NDVI (from satellite data), NDWI (from satellite data), soil information (e.g. surface soil moisture data from weather data and/or satellite data), and crop feature information such as average weight (e.g. kernel weight) and average dimensions (e.g. head and/or stem length).

The machine learning model is a classification model which can detect and classify multiple objects (i.e. crop features) in an image including the location of each object in the image. The model is trained on a dataset of crop images with known objects and attributes to predict what the object is based, at least in part, on the one or more image features extracted from the respective image.

The machine learning model may be or comprise a multivariate prediction model. The machine learning model may be or comprise any artificial neural network, such as a deep neural network or convolutional neural network. Additionally or alternatively, the machine learning model may be or comprise a regression model, a logistic regression model, a decision tree model, random forest model, a gradient boosting model, a naïve Bayes network, a k-nearest neighbour model, or a support vector machine. The machine learning model may be or comprise a data processing module, a software program or routine that processes the images.

In an embodiment, the machine learning model is trained on training data specific to the image resolution of the field camera and/or field images of the image data, as described in the fifth aspect. The training data may be generated from hyperspectral training images of crops in a controlled growth environment. The training data may be generated by the method of the fifth aspect.

The method may comprise extracting or calculating the one or more image features by applying one or more image feature detection algorithms or filters to the images, e.g. to one or more or all of the channels/bands of the respective image. The one or more image feature detection algorithms or filters may be configured to extract one or more image features from an image. For example, the one or more filters may be configured to generate a filtered and/or altered image from which one or more image features may be extracted or calculated/determined (e.g. from the altered pixel values). Alternatively or additionally, the one or more filters may be configured to extract the one or more image features directly from an image (e.g. from its pixel values).

The one or more crop feature attributes can be determined for each image/viewpoint, and each respective crop feature attribute can be combined, e.g. averaged and/or weighted, to provide one or more composite crop feature attributes that are more reliable and accurate than those determined from a single image/viewpoint, in a similar way to image stacking. As such, the step of determining, for each identified crop feature, one or more crop feature attributes may comprise, for each identified crop feature, combining each respective crop feature attribute extracted from each respective viewpoint to provide one or more composite crop feature attributes.

In an embodiment, each identified crop feature is geo-referenced, such that each crop feature has an absolute X,Y location relative to the field or earth's surface. If the images already form an orthomosaic map, each pixel in each image is already associated/registered with an X,Y location. If the crop features are identified from individual images which are not orthoimages or do not (yet) form an orthomosaic map, the X,Y location of each crop feature can be determined from the geo-location data of each respective image. As such, using an orthomosaic map to identify the crop features in each image is not essential, but can simplify the crop feature referencing.

The method may further comprise generating or updating a spatially resolved model or map of the identified crop features in the AOI. Each crop feature in the model is associated or tagged with an attribute vector comprising its respective one or more crop feature attributes. The model may comprise a two or three-dimensional (X, Y, Z) point cloud, where each two/three-dimensional point represents the (X, Y, Z) location of an individual crop feature which is associated/tagged with its attribute vector. The Z dimension may be relative, e.g. to the drone height AGL. The model may be or form a primary data structure for the crop monitoring method that can be stored, referenced and updated over time. In this way, the model is a multi-dimensional model with two or three dimensions in space (X, Y, Z), one in time, and one or more in crop feature attributes. Various crop monitoring outputs, such as feature maps, may be generated from the model data. Combining crop feature attributes determined from multiple viewpoints of the crop may reduce the noise in the model.

Generating the model may comprise extracting the geo-location information from each image and building a reference for the model. The orthomosaic map may form a reference frame for the model. Generating the model may further comprise populating the reference frame with two or three dimensional points representing the locations of individual crop features in the AOI, and associating/tagging each two/three dimensional point with its respective attribute vector.

The image data may comprise a plurality or series of images of the crops taken at different points in time, e.g. in the crop growth or cycle.

The model may comprise crop data, e.g. crop features and attributes, obtained at multiple different times to provide for temporal analysis. For example, the image data may be generated and/or capture the crop's state at a first time or date, and the method may further comprise repeating the steps for image data generated and/or capturing the crop's state at a different time or date. The method may comprise: receiving second image data containing a plurality of second images of crops in the AOI generated at a second time or date; identifying one or more crop features of each crop in each second image; determining, for each identified crop feature, one or more crop feature attributes; determining, based on the crop features and crop feature attributes, one or more crop monitoring outputs; and updating the model to include the crop features and crop feature attributes corresponding to the second time or date. The one or more crop monitoring outputs may be determined based at least on part on the crop features attributes determined at the first and second time.

Temporal stacking of crop feature attributes allows for monitoring the growth/state of crops throughout the crop cycle and/or the effectiveness of any interventions over time.

Using different viewpoint to identify crop features increases the fidelity of the model by stacking those different angles. Same can be said of stacking those different angles over time. For example, the image data may not include every viewpoint possible for a crop feature, however, if we miss a crop feature on one flight/mission/survey, but catch it on the next or previous, we can interpolate temporally to determine the crop features primary attributes. For example, if we detected a leaf of wheat on a drone flight mission, yet the next mission two weeks later we can't get the vantage point we need to detect it, we temporally interpolate that leaf (the leaf would grow depending on the crop stage of the wheat plant)

The method may comprise generating the image data using a satellite, or at least one camera mounted to a drone, or other image capture technology (e.g. digital camera or smart phone). Where generated by a drone, the method may further comprise providing flight control instructions to the drone to capture the image data. The method may comprise mapping each image to a different geolocation in the AOI, and/or generating an orthomosaic map of the AOI from the plurality images. Generating an orthomosaic map may comprise obtaining an array of overlapping images and stitching the array of images to form the orthomosaic map.

The orthomosaic map may contain a reference tile positioned within the AOI with a predefined size (e.g. one meter square) in order to adjust the images/map for variations in altitude and colour, as is known in the art. This may not necessary for satellite images which are taken from extremely high altitudes, but may be particularly useful for relatively low AGL drone images used to generate an orthomosaic map of the AOI. The reference tile, where present, provides a ground control point.

The method provides highly detailed and complete crop monitoring in an AOI using HR images. The AOI may be a whole field or a region of the field which is identified as being an AOI, e.g. a problem area and/or an area requiring further analysis.

In an embodiment, the method comprises receiving whole-of-field image data comprising one or more low resolution (LR) images of a field containing crops for monitoring, identifying an AOI in the field for subsequent HR imaging/mapping based on the one or more LR images, and receiving the HR image data of the AOI. Where there is one LR image of the whole field, the image may be an orthoimage. Where there is more than one LR image of the whole field, these may be an array of overlapping images, where each image is mapped to a different geolocation in the field. The overlapping LR images may form or be used to form/generate an orthomosaic map of the field. The method may comprise mapping each LR image to a different geolocation in the field, and/or generating an orthomosaic map of the field from the overlapping LR images.

The one or more LR images and/or HR images may be multi-spectral images, i.e. containing RGB and optionally IR bands.

Identifying the AOI may be based on one or more of: NDVI, VARI, NDWI, SSM index, and SOC level/index, BSI. Each of these indexes can be extracted from multi-spectral images, e.g. satellite images, as is known in the art. VARI requires RGB channels/bands. NDVI, NDWI, SSM index, SOC level/index and BSI require RGB and IR (NIR and SWIR) channels/bands, as is known in the art.

The whole-of-field image data may be obtained/generated by a satellite, manned aircraft, or a drone. Where obtained/generated by a drone, the LR drone images may be captured/generated at a constant altitude/height above ground level (AGL) in a drone survey, where the altitude AGL is greater than that used for generating the HR drone images, e.g. >100 m. The LR images have a lower resolution than the HR images. The method may comprise generating the LR image data using a satellite or at least one camera mounted to a drone. Where generated by a drone, the method may further comprise providing flight control instructions to the drone to capture the image data.

The method (including the machine learning model and/or one or more filters) can be implemented in software or software modules using the one or more processors or processing circuitry and machine readable medium or memory. The skilled person will however appreciate that the method and/or any software implemented element described herein, could equally be provided in hardware, firmware, or a combination of software, firmware and/or hardware.

According to a second aspect of the invention, there is provided a crop monitoring system, comprising processing device comprising processing circuitry and a machine readable medium containing instructions which, when executed on the processing circuitry, cause the processing device to perform the method of the first aspect. The processing circuitry may comprise one or more processors which may comprise the machine readable medium.

The machine readable medium may be configured to store the image data and any software for implementing the method. The system may comprise a drone, satellite and/or manned aircraft for generating the image data. The drone, satellite and/or manned aircraft may be in communication with, or configured to send and/or receive data to/from, the processing device. The drone may be configured to receive flight control instructions generated by the processing device for generating the image data of the AOI, and optionally send the generated image data to the processing device.

The system may further comprise one or more agricultural machinery units or vehicles for performing the interventions, and/or applying the one or more treatments to one or more regions of the AOI based on the one or more intervention instructions. The agricultural machinery units or vehicles may be configured to geo-position themselves and integrate the intervention instructions/data with its other functions, and may include any one or more of: tractors, crop sprayers (e.g. Chafer interceptors), seeders/planters/seed drillers (e.g. a Horsch Trailed Precision Seed Drill), any other form of precision agriculture machinery which utilises geo-positioning.

According to a third aspect of the invention, there is provided a machine readable medium containing instructions which, when executed on processing circuitry (i.e. one or more processors which may comprise the machine readable medium), cause the processing circuitry to perform the method according to the first aspect.

According to a fourth aspect, there is provided a processing circuitry and a machine readable medium containing instructions which, when run on the processing circuitry, cause the processing circuitry to perform the method according to the first aspect.

According to a fifth aspect of the invention, there is provided a method of generating training data for a machine learning model. The machine leaning model may be configured or used to determine crop feature attributes of crop features in images of crops. The images of crops may be field images generated by a field camera (imaging device) with a specific image resolution. The field camera may be a multi-spectral camera. The field camera may be a drone camera, or other image capture technology e.g. digital camera or smart phone. The method may comprise receiving image data containing a hyperspectral training image of crops generated/captured by or using a hyperspectral training camera. The crops may be grown and/or the hyperspectral images generated/captured in a controlled growth environment. The method may comprise generating one or more field camera-specific training images from the hyperspectral training image. The hyperspectral training image/camera may have a different image resolution to the field images/camera. The field camera-specific training images may have an equivalent image resolution to that of a field camera used to generate the field images. The method may comprise identifying one or more crop features of each crop, or a sub-set of crops, in the field camera-specific training images. The method may comprise labelling a sub-set of identified crop features with the one or more crop feature attributes. The method may comprise storing the labelled classified crop features in a database as a training data set for the machine learning model.

In this context, hyperspectral mean that each pixel of each image contains a large number of narrow spectral bands (i.e. a spectrum) covering a wide spectral range e.g. visible to NIR, as opposed to multi-spectral images that contain a relatively low number of broad spectral bands or colour channels such as red (R), green (G), and blue (B).

The machine learning model may be used for the crop monitoring method of the first aspect. The images of crops may be the images of crops of the first aspect.

The method advantageously produces training data adapted or adaptable for the specific imaging device or field camera used in the field for crop monitoring, as described in the first aspect. Classification of crop features and attributes is sensitive to the image resolution and spectral response of the field images input to the machine learning model and the training data is was trained on. The generation and use of camera-specific training data from high resolution hyperspectral images, e.g. with a pixel and spectral resolution that matches that of the field camera, can advantageously improve the accuracy and reliability of the machine learning model's ability to predict disease or other attributes affecting crop health that are essential to support intervention decisions in the crop monitoring process. The training data is essentially the best possible training data for the field camera used in the crop monitoring process. Further, the method can be repeated to generate any number of field camera specific training datasets from the same original high resolution hyperspectral images, to populate a unique database of training data, and develop bespoke machine learning models for the specific field camera.

By generating the training image data in a controlled growth setting or laboratory, the growth conditions of the crops are controlled and can be varied to develop specific crop feature attributes. Individual crops can be deliberately infected with a known disease, be supplied with different levels of nutrients, water and/or sunlight, and/or weeds or pests introduced, to controllably vary the crop health, yield and associated crop feature attributes. The growth conditions are ground control data that can be used to aid labelling.

The step of labelling may comprise determining, for each identified crop feature or at least a sub-set of identified crop features, one or more primary crop feature attributes. The primary attributes may be based on pixels or pixel attributes of the respective identified crop feature. Pixel attributes may include the pixel size, location and value in each spectral band. Pixel attributes of an identified crop feature may include a collective attribute of the pixels in the region(s) identified as a crop feature, such as a dimension (width, length), aspect ratio, principle axis, area, average pixel value (e.g. dominant colour), or other statistical property derived from the hyperspectral pixels making up the identified crop feature.

The one or more primary crop feature attributes comprise one or more geometric and/or spectral attributes derived from the pixels or pixel attributes of the respective identified crop feature. Geometric attributes may be derived from the size and location of pixels in the identified crop feature. The geometric attributes may include one or more of: location, dimension, area, aspect ratio, sub-feature size and/or count. Spectral attributes may be derived from the pixel values (intensities) in one or more spectral bands of pixels of the identified crop feature. The spectral attributes may include one or more of: dominant colour, RGB, red edge and/or NIR pattern, hyperspectral signature, normalised difference vegetation index (NDVI), and normalised difference water index (NDWI). Hyperspectral signature may be a spectrum, distribution or histogram of hyperspectral intensities. A pattern may be a collection of features such as edges or blobs.

The step of labelling may further comprise determining, for each identified crop feature or at least a subset of identified crop features, one or more secondary crop feature attributes. The secondary attributes may be based at least in part on the determined primary crop features attributes and/or ground control data for the crops and/or image. The ground control data may comprise known information collected from the controlled growth environment/setting including one or more of: crop type, disease type, weed type, growth conditions (e.g. level of sunlight, water, and/or nutrients/fertiliser provided), and crop age (e.g. relative age in the crop cycle). Labelling may be at least partly performed by an expert.

The step of generating one or more field camera-specific training images may comprise modifying the pixel values of the hyperspectral image based on the spectral response of the field camera. Modifying the pixel values may comprise determining a set of spectral filter weights associated with or for the specific field camera. The set of filter weights may comprise a filter weight for, and/or to be applied to, each spectral band of the hyperspectral image. The set of filter weights may be determined for, or associated with, a spectral band of the field camera. The set of filter weights may be determined based on the spectral response of the respective spectral band of the field camera. A set of filter weights may be determined for each spectral band of the file camera. Modifying the pixel values may further comprise applying the set of filter weights to the spectral bands of each pixel of the hyperspectral image. The step of generating one or more field camera-specific training images may further comprise generating the one or more field camera-specific training images from the modified pixel values of the hyperspectral image. The one or more field camera-specific training images may comprise one or more of: an RGB image, a near infrared image and a red-edge image.

The step of generating one or more field camera-specific training images may comprise re-sampling the hyperspectral training image to substantially match spatial and/or pixel resolution of the field camera. Resampling may comprise changing the number and size of pixels in the image. Re-sampling may be based on one or more equivalence parameters of the field camera, such as pixel resolution, focal length, field/angle of view, aperture diameter, and/or depth of field. Equivalence parameters are known for every camera, e.g. from the manufacturer's technical specification.

The image data may comprise a plurality or series of hyperspectral training images of the crops, each hyperspectral training image taken at a different point in time, e.g. in the crop growth or cycle. Each hyperspectral image may contain a time stamp. In this case, the method may comprise repeating the above steps for each point in time to generate a training set of labelled crop features at each point in time. For example, the method may comprise generating one or more field camera-specific training images from each hyperspectral training image in the time series, identifying, for each point in time, one or more crop features of each crop in the field camera-specific training images; and labelling, for each point in time, a sub-set of identified crop features with the one or more crop feature attributes including a respective time stamp.

In this way, the training data can include images of crops and/or crop features with labelled attributes for various stages of the crop's lifecycle. For example, training images may be taken over the course of several months, with several images taken each month.

Where there is a time series of hyperspectral training images, the method may further comprise applying one or more geometric and/or spectral corrections to the hyperspectral training images or the one or more field camera-specific training images associated with each different point in time to account for temporal variations in camera position and lighting conditions. This may comprise assigning one of the hyperspectral training images or field camera-specific training images associated with a given point in time as a reference image, and applying one or more geometric and/or spectral corrections to the other hyperspectral training images based on the reference image.

Applying one or more geometric corrections may comprise applying a geometric transformation to the other hyperspectral training images or the other field camera-specific training images associated different points in time to substantially match the spatial location and pixel sampling of the reference image. The geometric transformation may be based on the location size of one or more pixels of one or more ground control points in each image.

Applying one or more spectral corrections may comprise applying a white balance to, or altering the white balance of, the other hyperspectral training images or the other field camera-specific training images associated different points in time to substantially match the white balance of the reference image. The white balance may be applied or altered based on one or more pixels values of a ground control point (i.e. an object with known dimensions and colour) in each image. Applying or altering a white balance may comprise applying a global adjustment (increase or decrease) to the pixel values (intensities) in each spectral band of the hyperspectral images based on a comparison of one or more pixels values of the ground control point in each other image to the reference image.

Temporal analysis of image features in the training images and attributes involves comparing the training images and crop features in the images taken at different times. The above corrections produces a series of training images with comparable geometric properties and spectral signature so that they can be accurately aligned/overlaid, allowing for accurate quantitative temporal analysis.

Generating the one or more field camera-specific training images may be performed before or after the one or more geometric and/or spectral corrections.

The method may further comprise training a machine learning model using the field-camera specific training images and training data set. The method may comprise training a machine learning model to identify crop features and determine crop feature attributes of crop features in images of crops generated by a field camera using the field-camera specific training images and training data set. The machine learning model may be or comprise a deep or convolutional neural network. The trained machine learning model may be used in the crop monitoring method of the first aspect.

Each hyperspectral image in the time series may be taken from substantially the same position relative to the crops. The method may further comprise generating the image data by taking a hyperspectral image, or a series or plurality of hyperspectral images over a period of time, using a hyperspectral camera. The hyperspectral images may be taken with the hyperspectral camera in substantially the same position relative to the crops.

According to a sixth aspect of the invention, there is provided a non-transient machine-readable medium, containing program instructions that, when executed on processing circuitry, cause the processing circuitry to operate a machine learning model trained on the field camera-specific training data generated by the fifth aspect.

The machine readable medium referred to in any of the above aspects of the invention may be any of the following: a CDROM; a DVD ROM/RAM (including -R/-RW or +R/+RW); a hard drive; a memory (including a USB drive; an SD card; a compact flash card or the like); a transmitted signal (including an Internet download, ftp file transfer of the like); etc.

Features which are described in the context of separate aspects and embodiments of the invention may be used together and/or be interchangeable. Similarly, where features are, for brevity, described in the context of a single embodiment, these may also be provided separately or in any suitable sub-combination. Features described in connection with the device may have corresponding features definable with respect to the method(s), and vice versa, and these embodiments are specifically envisaged.

### BRIEF DESCRIPTION OF DRAWINGS

In order that the invention can be well understood, embodiments will now be discussed by way of example only with reference to the accompanying drawings, in which:

FIGS. 1(a) and 1(b) show illustrations of wheat crops and a wheat head respectively;

FIG. 2 shows an image of stripe rust and leaf rust on a leaf;

FIG. 3 shows a method of automated crop monitoring according to the invention;

FIG. 4 shows a method of generating image data;

FIG. 5 shows a schematic illustration of a drone imaging crops;

FIGS. 6(a) and 6(b) show a low resolution satellite image of a field indicating an area of interest and a corresponding normalised difference vegetation index (NDVI) map of the field, respectively;

FIGS. 7(a) and 7(b) show a high resolution image of crops and the same image highlighting detected crop features, respectively;

FIG. 8 shows a method of identifying crop features;

FIG. 9(a) shows an orthomosaic map of an area of interest;

FIG. 9(b) shows the same image in FIG. 9(a) overlaid with a crop feature density map;

FIG. 9(c) shows a zoom in of a region of the area of interest in FIG. 9(a);

FIG. 10 shows a schematic diagram of a system for implementing the method of FIG. 3;

FIG. 11 shows a method of generating training data;

FIG. 12 shows a schematic diagram of a controlled growth setting for generating training image data;

FIG. 13 shows an example training image of crops generated in a controlled growth setting with a map of individual crops in the image;

FIG. 14 shows a hyperspectral cube representation of an example hyperspectral training image;

FIG. 15 shows a composite image of different areas of crops generated from the training image of FIG. 13 after geometric correction with the map of individual crops in the image;

FIG. 16 shows example spectral response curves for the spectral bands of a field camera used for generating field camera-specific training images; and

FIGS. 17(a) to 17(c) shows example composite RGB, near infrared and red-edge field camera-specific training images generated from the training image of FIG. 13.

It should be noted that the figures are diagrammatic and may not be drawn to scale. Relative dimensions and proportions of parts of these figures may have been shown exaggerated or reduced in size, for the sake of clarity and convenience in the drawings. The same reference signs are generally used to refer to corresponding or similar features in modified and/or different embodiments.

### DETAILED DESCRIPTION

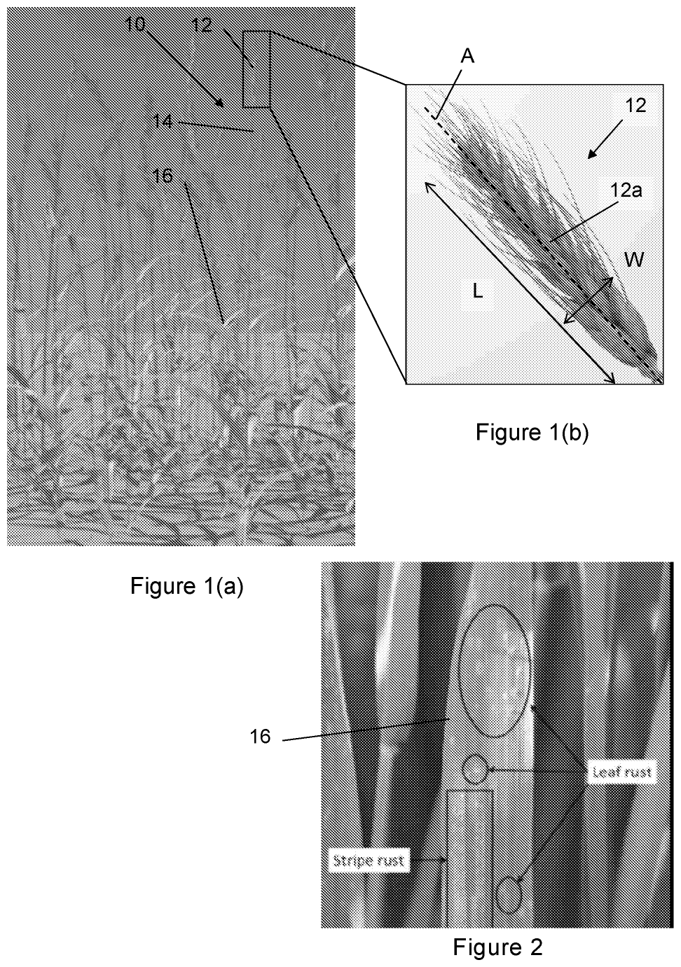

FIGS. 1(a) and 1(b) show example illustrations of crops 10 and crop features 12, 14, 16 and sub-features 12a which in this example are wheat plants 10, with heads 12, stems 14, leaves 16, and kernels 12a (grains/seeds). The crop features are inspected by farmers to monitor crop health, loss and yield. A crop feature 10 may have several attributes that correlate with crop health, loss and yield, and which a farmer can look for to base various conclusion and decisions on. For example, and pests such as aphids and mites can be detected upon close inspection of crop features. In addition, many diseases manifest as visible deterioration on the crop features that can be detected by experienced farmers at a relatively early stage, such as stripe rust on leaves 16 shown in FIG. 2. Also, the number of kernels 12a can be counted. By way of example, a wheat head 12 may have 25-50 kernels 12a depending on the health and nutrition of the crop. A high yielding crop of wheat may have 45-50 kernels per head, but this is reduced if nitrogen supply is limited. As such, a farmer can visually inspect the number of kernels 12a to base a conclusion that nitrogen supply is limited or yield is high. Based on detection of disease, pest, weeds, and/or poor health/yield the farmer can intervene by applying one or more treatments to the crops. e.g. fungicide, pesticide, weed killer, nitrogen etc. However, this manual process has several drawbacks: it is extremely time consuming; it is not feasible to inspect every crop so in practice only small sample or areas of crops are inspected and results are extrapolated across the whole field; and results can vary depending on the knowledge and experience of the farmer doing the crop monitoring.

The present invention automates and substantially improves upon manual crop inspection by processing and analysing a large number (typically hundreds or thousands) of aerial images of an area of interest (AOI) containing crops using computer vision and machine learning techniques to automatically detect and classify and analyse crop features and extract their attributes in a consistent manner throughout the AOI, and provide various quantitative outputs that a farmer can use to efficiently monitor and maintain the crops, such as accurate crop yield predictions, crop population and dimension statistics, crop-loss/disease diagnosis, spatially resolved maps of crop attributes and intervention instructions, as will be described in more detail below.

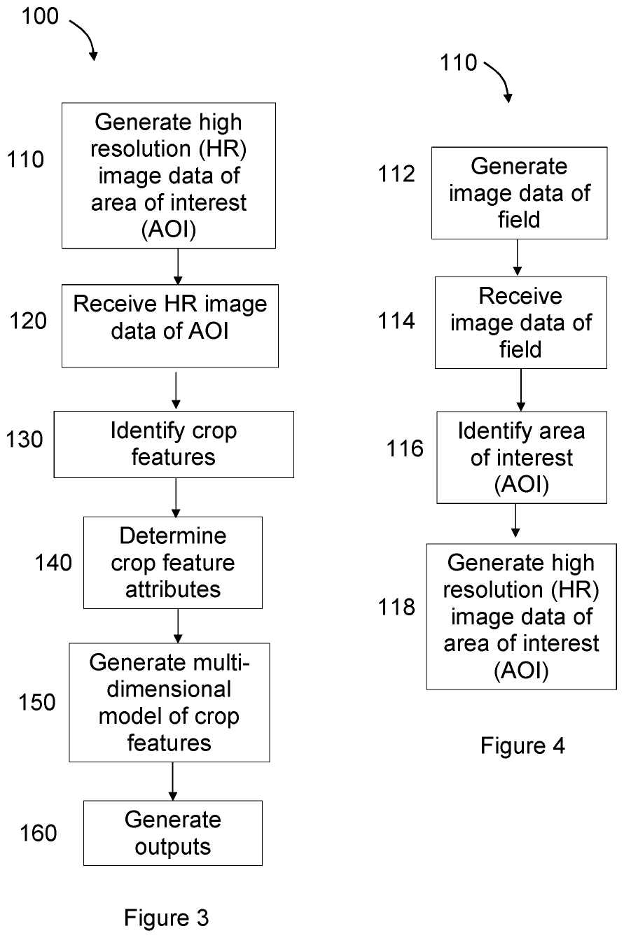

FIG. 3 shows an exemplary method 100 of automated crop monitoring according to an embodiment of the invention.

In step 120, image data containing a plurality of images I_{HR} of crops the AOI is received or retrieved, e.g. from a storage medium or database. The images I_{HR} are high resolution (HR) digital multi-spectral aerial images. In this context, multi-spectral means that each HR image I_{HR} comprises at least red, green, blue (RGB) colour channels, and may also include one or more infrared (IR) channels. Each HR image I_{HR} captures a different region of the AOI with sufficient pixel resolution to resolve individual crops 10 and crop features 12, 14, 16 in it, and contains geo-location (i.e. X, Y, or longitude and latitude) and time/date metadata. As such, the HR images I_{HR} contain the necessary geo-location information to reference them to the AOI, to a particular crop and to each other. The HR images I_{HR} are spatially overlapping to ensure complete coverage of the AOI. The HR images I_{HR} may be referred to as “field” images generated by a “field” camera with specific image resolution and/or spectral response (e.g. of each band/channel).

The required pixel resolution (pixels per meter) of the HR images I_{HR} is dependent on the type and size of crop being monitored. It will be appreciated that the size of the crop feature will depend on the time in the crop cycle. In the case of wheat crops, a fully grown wheat head 12 typically has a length L of approximately 8-12 cm, and a width W of approximately 2 cm (see FIG. 1(b)), with kernels approximated 5-10 mm in size. In this case, the HR images I_{HR} should have a pixel size of less than 10 mm, and preferably less than 2 mm if sub-feature recognition is required.

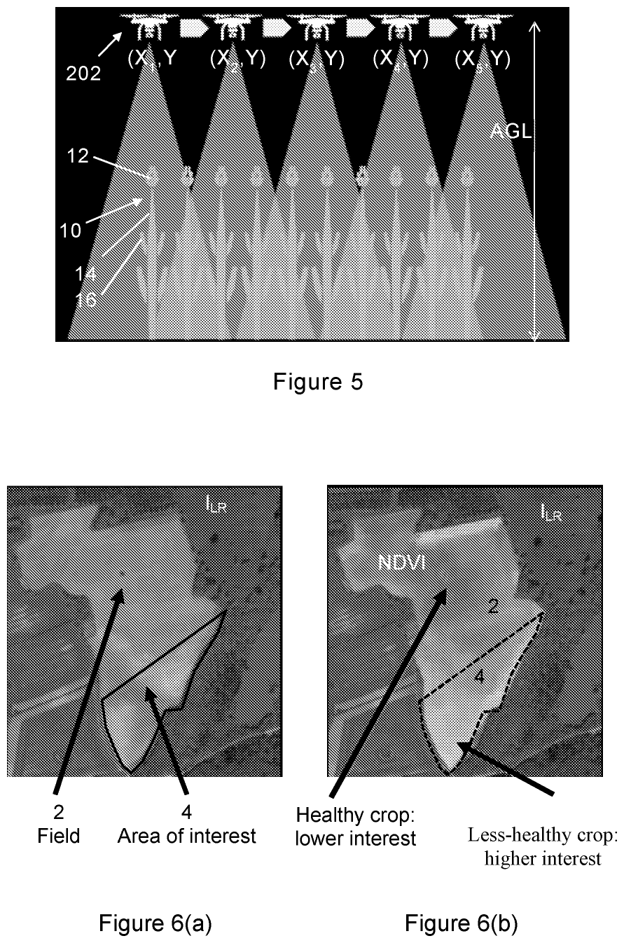

In an embodiment, the HR images I_{HR} are generated/captured by a camera mounted to an unmanned aerial vehicle (UAV) 202 or drone 202 (i.e. “drone” images), as illustrated in FIG. 5. The drone images are captured/generated at a constant altitude/height above ground level (AGL) in a drone survey of the AOL. The altitude AGL for generating the HR drone images I_{HR} is in the range 5 m to 50 m, and preferably 10-20 m. It will be appreciated that for a given camera pixel resolution, image resolution is increased at lower altitudes AGL. Each image I_{HR} is captured with the drone 202 in a different location in the AOI such that the images form an array whereby adjacent images overlap. In an embodiment the image overlap is at least 50% (50% in FIG. 5) to ensuring that each crop 10 is captured in multiple images, each from a different viewpoint. For example, where the array is a 2D array and the overlap is 50%, a crop in the AOI is imaged four times by the drone (with fixed camera orientation), where each image is taken when the drone is in a different position relative to the crop. As such, each image of the crop 10 is taken from a different position, providing different viewpoints/angles of the crop 10 from different sides. Imaging a particular crop 10 from multiple viewpoints increases the amount of input data per crop 10 increasing the reliability of the determined crop attributes (the same attributes can be determined from each viewpoint and combined), as described in more detail below. Alternatively, satellite images with the required resolution can be used (not shown).

The image data may initially be stored in an on-board memory of the drone 202 and later transferred for storage, processing and analysis. Once the image data is received, the geolocation information from each HR image I_{HR} is extracted and the images I_{HR} are mapped/referenced to the AOI. Where the image data comprises video data, the plurality of HR images I_{HR} are first extracted as image frames/image stills from the video data. In an embodiment, an orthomosaic map of the AOI is generated by stitching the HR images I_{HR} together, as is known in the art. In the orthomosaic map, each pixel is referenced to a geo-location. The images I_{HR} may then be adjusted for variations in altitude and colour scaling, as is known in the art. For example, a reference tile with a known size (e.g. 1 m×1 m) can be positioned within the AOI to appear in the orthomosaic map, and the images I_{HR} can be normalised based on a reference tile's apparent size and RGB composition in image.

The method 100 may include the step 110 of generating the image data. Where the images I_{HR} are drone images, step 110 may comprise providing flight control instructions to a drone 202 to survey the AOI at a specified altitude AGL and image overlap.

The AOI may be a whole field or a region of a field. Where the AOI is a region of a field, step 110 may comprise identifying an AOI. The AOI may be identified empirically based on historical data, for example, the locations of historical crop loss events (not shown). Alternatively, it may be identified by remote image sensing techniques including, but not limited to, normalised difference vegetation index (NDVI), visual atmospheric resistance index (VARI), normalized difference water index (NDWI), surface soil moisture index (SSMI), and soil organic-carbon index (SOCI). Each of these indexes can be extracted or derived from multi-spectral or hyperspectral satellite images, as is known in the art. VARI uses RGB channels/bands, whereas NDVI, NDWI, SSMI and SOCI use RGB and IR channels/bands. NDVI is a basic quantitative measure of live green plants, determined from the ratio NDVI=(red−NIR)/(red+NIR) where red and NIR are the red and near-infrared (NIR) light spectral channels of the image, and ranges from −1 to 1. NDVI emphasises the green colour of a healthy plant and is commonly used as an indicator of chlorophyll content in several different types of crops, including corn, alfalfa, soybean, and wheat. NDVI is therefore used as a course indication of crop health, 1 being healthy. VARI provides similar information to NDVI but is less sensitive to atmospheric effects, allowing for vegetation to be estimated in a wide variety of environments. NDWI is correlated to the plant water content, and is therefore a good indicator of plant stress. NDWI is determined from the ratio NDWI=(NIR−SWIR)/(NIR+SWIR) where NIR and SWIR are the NIR and shortwave-infrared (SWIR) light spectral channels of the image, and ranges from −1 to 1. SSMI provides information on the relative water content of the top few centimetres of soil, describing how wet or dry the soil is in its topmost layer, expressed in percent saturation. It therefore provides insights in local soil conditions. SSMI is described in “Retrieving soil moisture in rainfed and irrigated fields using Sentinel-2 observations a modified OPTRAM approach” by A. Ambrosone et al. International Journal of Applied Earth Observation and Geoinformation 89, 102113 (2020). SOC refers to the carbon component of organic compounds in soil, which contribute to nutrient retention and turnover, soil structure, moisture retention. SOCI is described in “Estimating soil organic carbon in cultivated soils using test data, remote sensing imagery from satellites (Landsat 8 and Plantscope). and web soil survey data” by M. Halil Koparan, Thesis, South Dakota State University (2019). The values of and spatial variations in any of these indexes across a field can therefore be used to identify an AOI.

FIG. 4 shows example steps of identifying an AOI using remote image sensing and generating the HR image data. In step 114, image data containing one or more multi-spectral images I_{LR} of a field is received. The image(s) I_{LR} used for AOI identification may be relatively low resolution (LR) compared to the HR images I_{HR} described above. In an embodiment, the LR image data contains a single whole-of-field satellite image (see FIG. 6(a)). Alternatively, the image data may contain a plurality of overlapping drone or satellite images from which an orthomosaic map of the field is generated. Where LR drone images are used, these are captured from a greater height AGL than the HR drone images, e.g. AGL>100 m. In step 116, the AOI is identified based on one or more of VARI, NDVI, NDWI, SOCI and SSMI maps of the field calculated from the spectral bands/channels of the LR images I_{LR}. For example, a predefined threshold may be applied to the VARI, NDVI, NDWI, SOCI and/or SSMI maps to indicate or identify areas of less healthy crops. In step 118, image data containing the HR images I_{HR} of the AOI is generated e.g. by drone survey, as described above.

FIG. 6(a) shows an example satellite RGB image of a field 2 of crops 10 in RGB (colour) obtained from the Sentinel-2 satellite 201. FIG. 6(b) shows the same satellite image overlaid with the NDVI map of the field 2. Regions of healthy and less healthy crops can be identified from the high and low NDVI values as shown and used to identify the AOI 4 for HR imaging in step 118.

In step 130, one or more crop features 12, 14, 16 of each crop in each HR image I_{HR} are identified using a using a machine learning model trained on a dataset of crop images to detect and classify the one or more crop features in the respective HR image I_{HR}. In an embodiment, the machine learning model comprises a convolution neural network (CNN) and leverages Google Vision, Opencv, and Scipy Libraries.

Identifying the crop features 12, 14, 16 in an image I_{HR} involves extracting and/or calculating one or more image features from the respective image I_{HR} using one or more feature extraction filters. Image features may be specific structural features in the image itself, such as points, edges or objects, or statistical information calculated or determined from the pixels values, such as averages, standard deviations, or distributions. The one or more image features may be low-level image features, e.g. related to pixel attributes. The one or more image features may be extracted from one or more, or all, of the channels/bands of the respective image I_{HR}. The one or more image features may comprise any one or more of: edges, corners, ridges, blobs, RGB colour composition, area range, shape, aspect ratio, and feature principle axis. In an embodiment, the one or more image features include at least edges.

The machine learning model takes the one or more image features as inputs, detects objects including the location of each object in the image and outputs a probability of each detected object being a crop feature belonging to one of the crop feature types (e.g. head 12, stem 14, leaf 16). The geo-location of each detected crop feature is determined in order to combine information on the same crop feature from different images (see below).

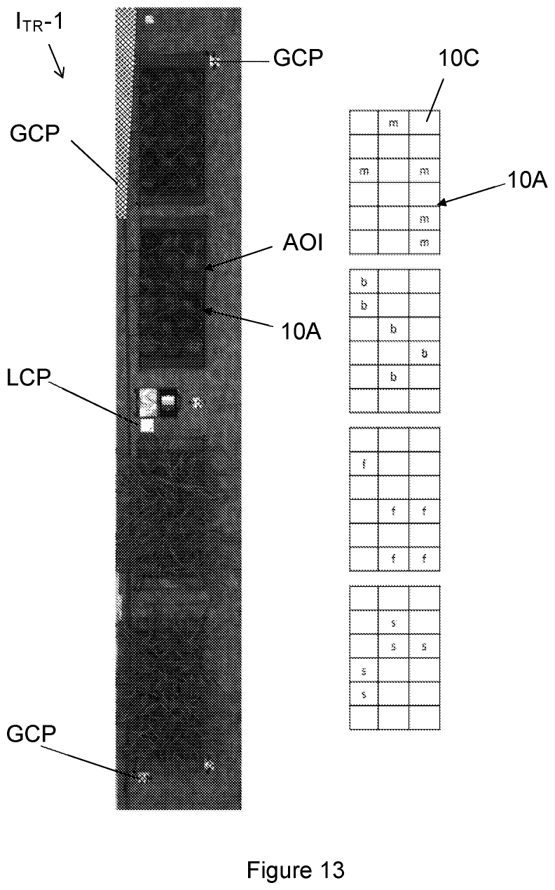

The general approach is to train the machine learning model using a dataset of crop images with known crop features and attributes which have been identified by an expert. Once trained, a new image can then be input to the machine learning model to detect and classify crop features and attributes in it. The machine learning model can be trained on historical training data, or where this is not present, using a subset of HR images I_{HR} from the image data of the AOI. Continual expert input is not needed. The model requires training for each new crop or feature, but once a critical mass of training data is achieved, further expert input is not necessary. In an embodiment, the machine learning model is trained on a training dataset generated specifically for the field camera used to generate the HR images I_{HR}, as described below with reference to FIGS. 13 to 17.

The image features may be extracted from the images using various known (and open source) image feature detection algorithms or filters. Many different feature detection algorithms are known in the field of image processing. By way of example only, edges can be extracted using a Canny or Sobel edge detection algorithms, blobs can be detected using a Laplacian of Gaussian (LoG) algorithm, and corners may be detected using a Harris corner detection algorithm, etc. Each filter typically represents a portion of code that can be run to extract the image feature(s) in question. As such, it will be appreciated that a separate filter may be configured to extract each separate image feature, or one or more filters may be configured to extract a plurality of images features.

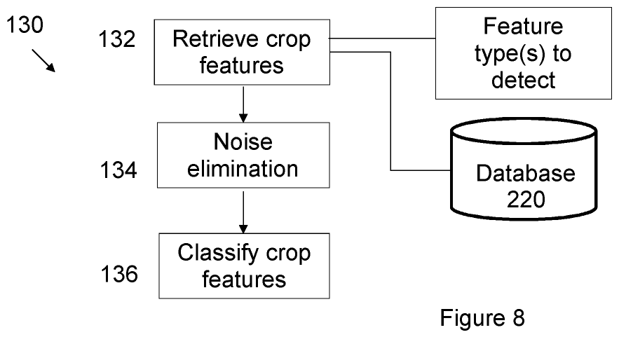

An example process flow for crop feature identification process 130 is shown in FIG. 8. In step 132, the type(s) of crops features to be detected are retrieved from a database 220. This may involve retrieving certain attributes associated with each crop feature, such as expected colour, dimensions etc. The database 220 may store the training images and cropped images of various crop features for use by the machine learning model. In step 134, noise in the images is removed. Noise elimination involves removing parts of the image are definitely not part of the crop feature to be detected based on information retrieved from the database 220. For example, blobs that do not fit to the dimensions of the crop feature can be removed, and parts of the image that show soil, which is brown and which does not match the expected colour of a crop feature can be removed (e.g. pixels set to zero). This reduces the noise in the image to increase the signal for feature detection in the machine learning model, e.g. CNN. In step 136, the machine learning model (e.g. CNN) is used to detect and classify the crop features in the image I_{HR}. This may involve processing the image I_{HR} using the one or more filters (e.g. at least an edge detection filter).

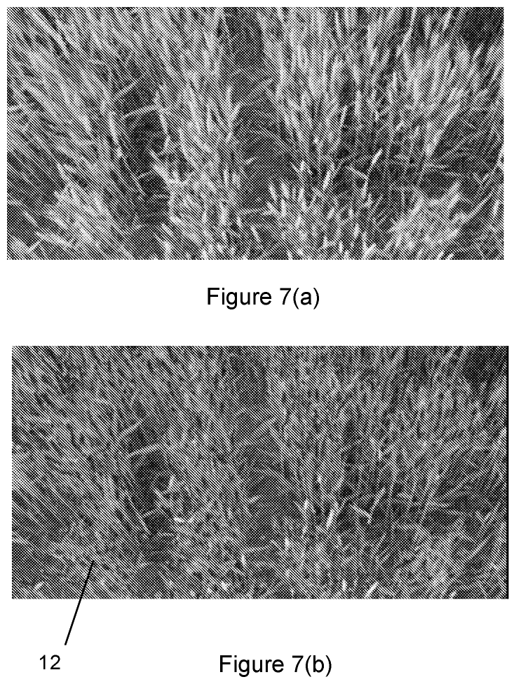

FIG. 7(a) shows an example input image I_{HR} containing wheat crops 10, and FIG. 7(b) shows the same image I_{HR} where the wheat heads 12 have been identified using the above process 130.

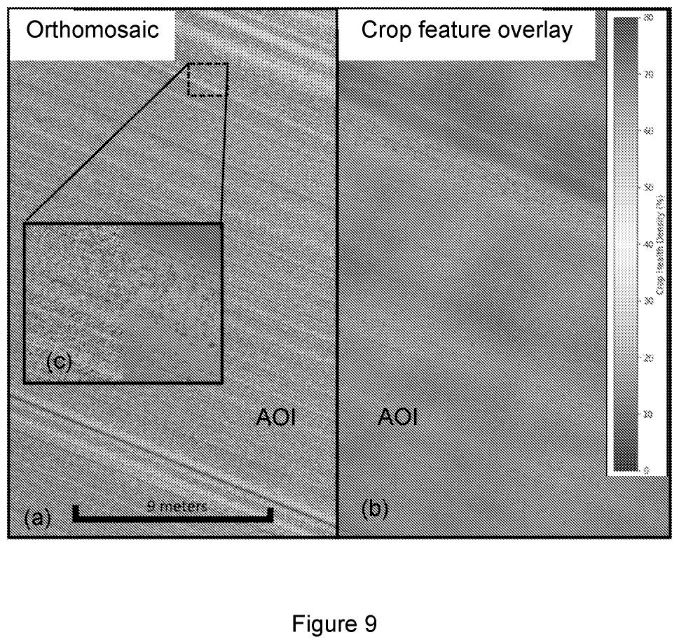

FIG. 9(a) shows an example geo-referenced orthomosaic map of an AOI containing wheat crops 10 that may be input to the above process 130. The orthomosaic map was generated from a drone survey at a height AGL of 12 meters. The pixel size is approximately 6-7 mm and the scale bar is 9 meters. In this case, the drone survey was performed at a relatively early tillering stage in the wheat crop cycle before heading (so there are no wheat heads 12), such that the crop features to be detected are stems 14 and leaves 16. In step 132, these features are retrieved from the database 220. FIG. 9(b) shows a feature density map overlaid on the orthomosaic map, produced from the crop features and locations obtained in step 136 (noise elimination was performed in step 134). The feature density map is the density of crop features per unit area, and this can be used to display “health density”, by combining the various crop feature attributes determined in step 140, described below. FIG. 9(c) shows the same (in the left and right panels) for a zoomed in region of the orthomosaic map in which individual wheat crops 10 can be distinguished.

In step 140, one or more crop feature attributes are determined for each identified crop feature. This may involve determined one or more primary, secondary and/or tertiary attributes.

Primary attributes are derivable directly from the image pixel attributes/values and/or extracted image features in the respective image I_{HR} and include, but are not limited to any one or more of: location, dominant colour, dimension(s), and sub-feature 12a size and/or count. The location of each crop feature may be determined, at least in part, using geolocation data/information of each respective image in the image data. Where the images form an orthomosaic map of the AOI, the locations of crop features can be determined directly from the pixel values.

Secondary attributes are derivable/determined indirectly from the images I_{HR} using a machine learning model similar to process 130. Secondary attributes include, but are not limited to any one or more of: diseased and disease type, pest-ridden and pest type, weed-ridden and weed type, healthy, and unhealthy. In this case, the machine learning model, which may be the same machine learning model used for feature classification 130, is trained on a dataset of images of crop features (e.g. stored in the database 220) with known secondary attributes, such as known diseases and pests. For example, the machine learning model may be trained to detect various diseases such as septoria, stripe rust and leaf rust, various pests such as aphids, mites, orange wheat blossom midge (OWBM), locusts, and various weeds such as black grass. A crop feature may be classed as healthy if no disease, pests or weeds are detected.

Tertiary attributes are derived using additional input data, e.g. satellite images and ground data, and include any one or more of: NDVI, soil information, and weight.

The one or more crop feature attributes are determined for each image/viewpoint of a particular crop, resulting in multiple values for each respective attribute for each crop feature. These respective crop feature attributes are then combined or stacked, e.g. averaged and/or weighted, to provide composite crop feature attributes that are more reliable and accurate than those determined from a single image/viewpoint, similar to image stacking.

In step 160, a spatially resolved model of the crops in the AOI is generated based on the identified crop features and attributes. The crop model is a three-dimensional (3D) point cloud model, where each 3D point represents the location of a crop feature in the AOI, which is tagged or associated with a respective attribute vector containing all the determined attributes. The Z-component of each 3D point may be relative. Relative Z can be determined via the drone using an on-board range sensor (e.g. LIDAR) or altitude sensor (e.g. barometer), if available. If these instruments are not available, a digital elevation model (DEM) is created from the highest resolution satellite image data of the AOI available. The crop model is generated from the plurality (typically hundreds or thousands) of HR images I_{HR} of crops taken from different angles. The reliability of the model data, i.e. detected crop features and attributes, is increased by stacking several feature attributes from different viewpoints to decrease noise (as described above), while not disturbing the primary signal (feature). A reference frame for the model is built from the extracted geo-location information of each image. Where an orthomosaic map has been generated from the HR images I_{HR}, this can form the reference frame for the model. The reference frame can then be populated with 3D points for the individual crop features in the AOI using their determined locations (primary attribute). Each 3D point is associated/tagged with its respective attribute vector.

The model is the primary data structure for the crop monitoring method 100 that can be stored (e.g. in database 220), referenced and updated over time. Where the crop model is already generated (e.g. based on the crop monitoring method being performed at an earlier time) step 160 may instead comprise updating the crop model with the (newly) identified crop features and attributes. For example, the model can comprise crop features and attributes for an AOI extracted from image data generated at multiple different times to provide for temporal analysis. The temporal data can also be used to further increase the reliability of detected crop features and attributes by stacking the feature attributes obtained at different times to decrease noise (as described above). For example, the image data may be generated and/or may capture the crop's state at a first time or date, and the method may be repeated for additional image data generated and/or capturing the crop's state at a different time or date, and that data can be added to the crop model. Each 3D point may then be tagged with an attribute matrix describing the attributes at different times.

Using different viewpoints to identify crop features increases the fidelity of the model by stacking those different angles. Same can be said of stacking those different angles over time. For example, the image data may not include every viewpoint possible for a crop feature, however, if we miss a crop feature on one flight/mission/survey, but catch it on the next or previous, we can interpolate temporally to determine the crop features primary attributes. For example, if we detected a leaf of wheat on a drone flight mission, yet the next mission two weeks later we can't get the vantage point we need to detect it, we temporally interpolate that leaf (the leaf would grow depending on the crop stage of the wheat plant).

In step 160 one or more crop monitoring outputs are generated, at least in part, based on the crop features and crop feature attributes. The one or more crop monitoring outputs include one or more of: a crop feature population count, a volumetric crop yield prediction, one or more intervention instructions, and various spatially resolved crop feature or sub-feature attribute maps or meshes. The maps may include a crop feature population density map, dimensions map (wheat head 12 length or stem 14 length), sub-feature count map (e.g. number of kernels 12a per crop), crop loss and/or disease map. The crop monitoring outputs may be generated from the data held in the crop model.

In one example, a volumetric crop yield prediction can be generated based on the dimensions of each crop feature 12 and/or sub-feature 12a, the crop feature 12 count and/or sub-feature 12a count, and weight information such as average weight per kernel 12a.

The one or more intervention instructions may comprise instructions to apply one or more treatments to one or more regions of the AOI, e.g. applying water, seeds, nutrients, fertiliser, fungicide (e.g. for disease treatment), and/or herbicide (e.g. for weed killing) based on the data in the crop model. The one or more interventions may include identifying high-productivity regions in the AOI combined with identifying regions for targeted seed planting, targeted fertilising (e.g. nitrogen, phosphorus, potassium) and other yield improvement measures, herbicides (e.g. targeted black grass spray interventions) and other weed killing interventions, targeted fungicides and other disease treatments and mitigation interventions, water stress/pooling monitoring with targeted interventions; as well as monitoring the effectiveness of any of these interventions over time. For example, regions of the AOI where diseased crops are detected may be targeted for disease treatments.

In an embodiment, the instructions may be machine integrated instructions for one or more agricultural machinery units or vehicles to apply the one or more treatments to the one or more regions. Farm machinery is increasingly automated, and satellite guided (GPS, GNSS, etc), hence the machinery understands where it is located in that field or AOI. Some machinery is able to follow a predetermined track on autopilot (e.g. John Deere's fully-autonomous tractor concept). The intervention instructions can include a shapefile which integrates with this existing farm machinery in such a way that the tractor, variable rate fertiliser applicator, or any other form of farm machinery, understands where it is in the field relative to the shapefile, and what action it is meant to perform at that location (e.g. spray a certain volume of nitrogen, herbicide, fungicide).

The method 100 can be used to develop optimal relationships between extracted primary crop feature attributes and derived secondary or tertiary attributes such as overall crop health. For example, currently NDVI is typically used to correlate to crop yield, with an error margin of approximately +/−33%. By contrast, the method 100 relies primarily on feature recognition to predict yield, which is more direct and accurate.

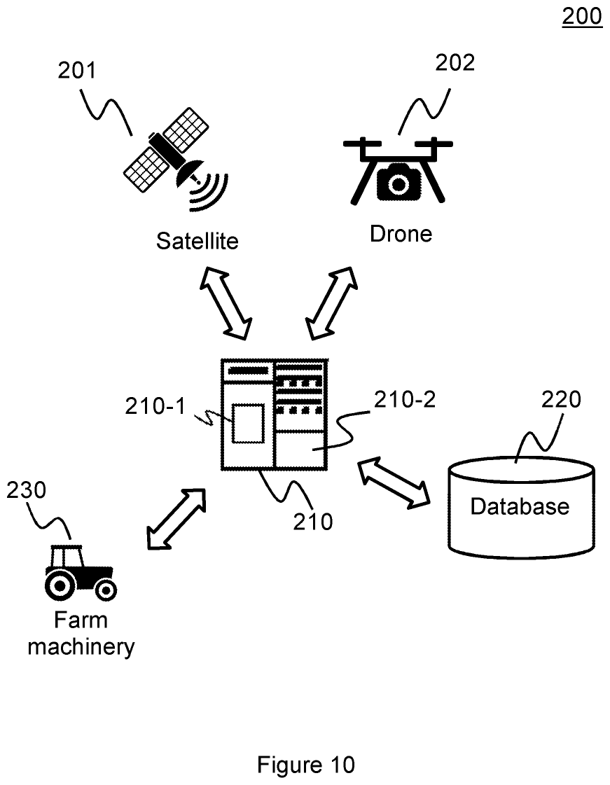

FIG. 10 shows an exemplary system 200 for implementing the method 100 described above. The system 200 comprises: one or more processing devices 210; a database 220 in communication with the processing device(s) 210; and a drone 202 for generating the HR images I_{HR} of the AOI 4. The system 200 may also comprise a satellite 201 for providing LR and HR images of the field 2 and/or AOI 4, and/or farm machinery 230 for applying treatments to one or more regions of the AOI 4 based on one or more of the outputs generated by the method 100. Alternatively, the system 200 may comprise any suitable imaging system for generating the HR image data, such as a smart phone or digital camera (not shown). Steps 120-160 are computer-implemented data processing steps which can be implemented in software or one or more software modules using the one or more processing devices 210. The system 100, 200 can be implemented in multiple platforms, such as a web page interface or an app interface.

The processing device(s) 210 may include a one or more processors 210-1 and a memory 210-2. The memory 210-2 may comprise instructions that, when executed on the one or more processors 210-1, cause the one or more processors 210-1 to perform at least steps 120-160 described above. The one or more processors 210-1 may be single-core or multi-core processors. Merely by way of example, the one or more processors 210-1 may include a central processing unit (CPU), an application-specific integrated circuit (ASIC), an application-specific instruction-set processor (ASIP), a graphics processing unit (GPU), a physics processing unit (PPU), a digital signal processor (DSP), a field programmable gate array (FPGA), a programmable logic device (PLD), a controller, a microcontroller unit, a reduced instruction-set computer (RISC), a microprocessor, or the like, or any combination thereof.

The processing device(s) 210 may be implemented on a computing device or a server. The server may be a single server, or a server group. The server group may be centralized, or distributed (e.g., server 210 may be a distributed system). The server 210 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

The database 220 may store data and/or instructions, such as the HR and LR image data, the crop model, and training images for the machine learning models. Although shown as separate from the processing device(s) 210 in FIG. 10, the processing device(s) 210 may comprise the database 220 in its memory 210-2. The database 220 and/or the memory 210-2 may include a mass storage, a removable storage, a volatile read-and-write memory, a read-only memory (ROM), or the like, or any combination thereof. Exemplary mass storage may include a magnetic disk, an optical disk, a solid-state drives, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc.

Exchange of information and/or data between the processing device(s) 210, satellite 201, drone 202, and database 220 may be via wired or wireless network (not shown). Merely by way of example, the network may include a cable network, a wireline network, an optical fiber network, a tele communications network, an intranet, an Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, or the like, or any combination thereof.

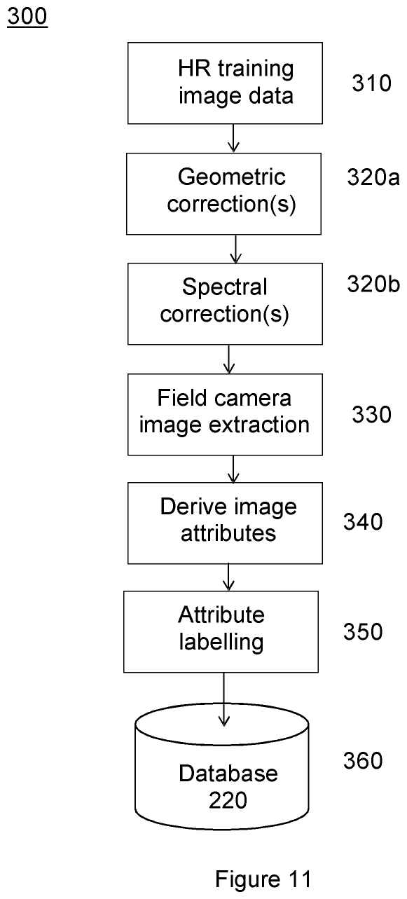

FIG. 11 shows an exemplary method 300 of generating a database 220 of training data for training the machine learning model according to an embodiment of the invention. The training data can include training images of crops 10 and/or crop features 12, 14, 16 obtained at various stages of the crop's lifecycle. Each crop feature 12, 14, 16 is labelled or tagged with one or more crop feature attributes (primary, secondary and/or tertiary attributes) used for classifying the crop feature. The crop feature attributes can include one or more primary, secondary and/or tertiary attributes, as described above. In this case, all the crop feature attribute labels are derived from high resolution hyperspectral training images I_{TR} while the crops are growing in a controlled environment or laboratory under control growth conditions, as described below. In particular, the method 300 produces training data adaptable for the specific imaging device or camera used in the field for the crop monitoring process 100 (referred to hereafter as the “field camera”). For example, a specific field camera will have a specific image resolution (e.g. pixel resolution, spatial resolution and spectral resolution/responsivity) depending on the type of camera it is. It may also have a specific focal length and other equivalence parameters affecting the resulting images. As such, the same crop imaged by two different field cameras under identical conditions (e.g. same lighting conditions and same position relative to the crop) may look slightly different, e.g. in RGB and/or NIR channels, which may yield potentially different classification outcomes when using a machine learning model trained on or utilising the same training data. As such, the generation and use of camera-specific training data, e.g. with a pixel and spectral resolution that matches that of the field camera, can improve the accuracy and reliability of the classification and crop monitoring process. In addition, the method 300 applies the same geometric and spectral pre-processing to each lab-based training image to reduce noise and make them directly comparable for temporal analysis. Comparing and iterating through a large number of noisy training data is already an issue with known methodologies, which would be compounded with different effects from lab and field conditions. As such, each lab training dataset generated from the method 300 becomes quite unique.

In this context, hyperspectral mean that each pixel of each image contains a large number of narrow spectral bands (i.e. a spectrum) covering a wide spectral range e.g. visible to NIR, as opposed to multiple-spectral images that contain a relatively low number of broad spectral bands or colour channels such as R, G, B and optionally NIR.

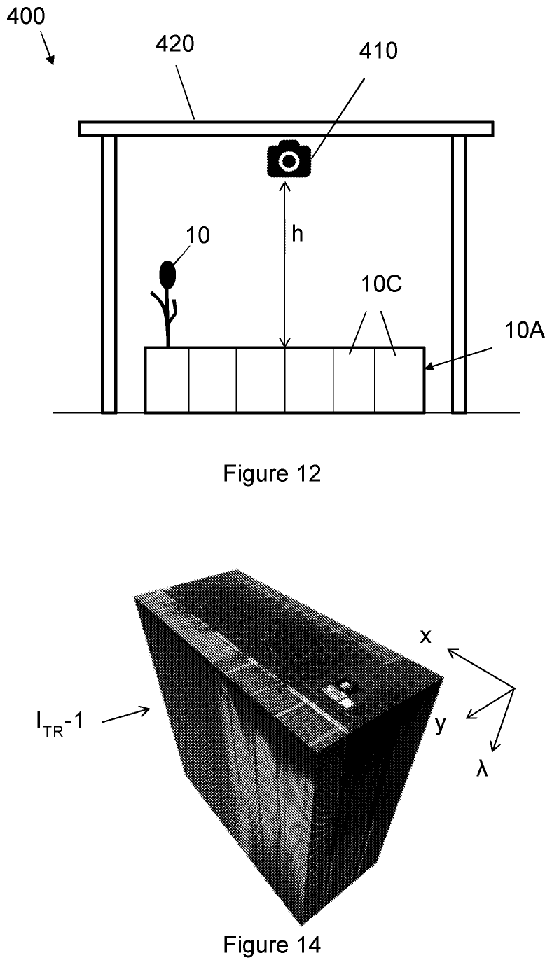

To understand the highest level of feature analysis it is necessary to start with and analyse the highest possible resolution image data. In step 310, high spectral, temporal, and spatial resolution training images I_{TR} of crops are generated, obtained or collected. The training images I_{TR} are obtained in a controlled setting or environment 400, such as a laboratory, using a HR hyperspectral imaging device or camera 410 (referred to hereafter as a training camera 410), as shown schematically in FIG. 12. In the controlled setting 400, crops 10 are grown under controlled conditions and training images I_{TR} are captured from a fixed position above the crops 10 at different times throughout the crop cycle. This produces a set of training images I_{TR} for the crop 10. For example, training images I_{TR} may be taken over the course of several months, with several images taken each month. The growth conditions of the crops 10 are controlled and can be varied to develop specific crop feature attributes. Individual crops 10 can be deliberately infected with a known disease, and/or be supplied with different levels of nutrients, water and/or sunlight to controllably vary their health, yield and associated crop feature attributes. As such, step 310 may comprise a step of growing crops 10 in a controlled setting wherein at least some of the crops 10 have one or more known crop feature attributes, and/or controlling one or more growth conditions of the crops 10.

In the example controlled setting 400 of FIG. 12, the training camera 410 is mounted to a support structure or gantry 420, allowing the position of the training camera 410 relative to the crops 10 to be controlled and fixed over long periods of time. The height h of the training camera 410 above the crops 10, which is equivalent to the height above ground level (AGL) in field data such as a drone survey, is relatively low compared to a typical field data to maximise the spatial resolution of the training images I_{TR}. In an embodiment, the height h is less than 1 m. The setting 400 may also include one or more UV lights to simulate sunlight and control crop growth (not shown). Individual crops 10 are grown in predefined positions in an array 10A of pots or containers 10C.

Depending on the size of the array 10A and/or number of arrays 10A, the training camera 410 can be mounted to a motorised translation stage (not shown) to adjust its position (x and/or y) and capture images of the different areas or arrays 10A at the same height h sequentially. In one example, the training camera can be used to capture a panoramic scan of the one or more arrays 10A at a constant height h. Alternatively, a plurality of training cameras 410 can be used to capture images I_{TR} of different areas of an array 10A or different arrays 10A. In this case, identical training cameras 410 are positioned at the same height h and capture images simultaneously. Multiple images from the same or different training cameras 410 can be stitched together to generate an orthomosaic map of the crops 10 at the given point in time, as described above.

FIG. 13 shows an example raw RGB training image I_{TR}-1 of a set of four 6×3 arrays 10A containing wheat crops 10 taken. The image I_{TR}-1 was taken as a panoramic scan. There are 72 containers 10C divided between four arrays 10A. Each array 10A contains a different category of crop 10, as indicated by the letters m (mixed blackgrass and healthy wheat), b (blackgrass), f (fusarium head blight), and s (septoria).

Each training image I_{TR} comprises x-y pixel data in a plurality of a plurality of narrow spectral bands. For example, a hyperspectral camera 410 can contain 973 discrete spectral bands spanning a broad spectral range e.g. λ˜400-1000 nm, whereas a multi-spectral camera 410 typically contains 3-5 bands such as red (λ˜650 nm), green (λ˜560 nm), blue (λ˜450 nm), red edge (λ˜730 nm) and NIR (λ˜840 nm). Where hyperspectral images are acquired, each training image I_{TR} can be represented by a three dimensional hyperspectral data cube I_{TR}(x, y, λ), where x and y are the two spatial dimensions of the scene and λ is the spectral dimension comprising a range of wavelengths, as shown in FIG. 14. Different spectral bands reveal different features of a crop and therefore contain different information relevant to identifying and classifying crop features and attributes. For example, certain diseases such as septoria are more visible in the NIR than in the visible wavelength range (RGB). The different spectral bands in the image data I_{TR}(x, y, λ) can be analysed individually or combined to produce specific types of composite spectral images. As such, various different types of spectral images can be derived from each hyperspectral training image I_{TR}(x, y, λ), including, but not limited to, traditional RGB images, red edge images (λ˜730 nm) and NIR images (λ˜840 nm), as well as the more advanced remote sensing image indices described above such as NDVI, VARI, NDWI, SSMI and SOCI.

Temporal analysis of image features in the training images I_{TR}(x, y, λ) involve comparing training images and crop features in the images taken at different times. As such, any meaningful temporal analysis requires each training image I_{TR}(x, y, λ) in the set (which is acquired at a different time) to have comparable geometric properties and spectral signature so that they can be accurately aligned/overlaid. Because each training image I_{TR}(x, y, λ) in the set is acquired at different time, with a time interval between images on the order of days, the position of the training camera 410 relative to the crops 10 (e.g. its height h, lateral x, y position, and/or tilt) is subject to possible drift and the lighting conditions (e.g. natural and/or artificial light) in the controlled setting 400 may vary between images. As the spatial resolution of the training images I_{TR} is so high, any misalignment and/or change in lighting conditions introduces error the analyses performed the proceeding steps. In particular, it is difficult to quantitatively compare image features taken at different times or crop features in the same image if the crop features or images taken at different times have different lighting conditions. As such, any changes in geometric and/or spectral characteristics are corrected for in steps 320a and 320b.

In step 320a, the set of raw training images I_{TR}-1 are processed to apply geometric corrections. Geometric corrections involve reorienting and stretching the training images to ensure each training image has the same pixel size and shape. Step 320a comprises assigning one of the training images of the set as a reference image, and adjusting each other training image to the spatial location and pixel sampling of the reference image. This involves determining or identifying one or more ground control point (GCP) locations in each training image, and applying a transformation (e.g. an affine transformation) to adjust the pixel locations and sampling to match that of the reference training image based on the location and size of one or more pixels at a GCP, as is known in the art. Example GCPs are shown in FIG. 13, which are checkered reference tiles with known dimensions.

In step 320b, the set of training images are processed to apply a spectral correction. Spectral correction involves applying a white balance or a global adjustment to the spectral intensities (increase or decrease) of the training images so that all the training images have the same whiteness level (also known as colour balance, grey balance or neutral balance). Step 320b comprises adjusting the whiteness level of each other training image to match the whiteness level of the reference training image. This involves identifying one or more lighting control point (LCP) locations in each training image and adjusting the whiteness level to match that of the reference training image based on the spectrum of one or more pixels at the LCP, as is known in the art. A LCP is a reference tile with neutral colours such as white and grey. Step 320b can be performed before or after step 320a.

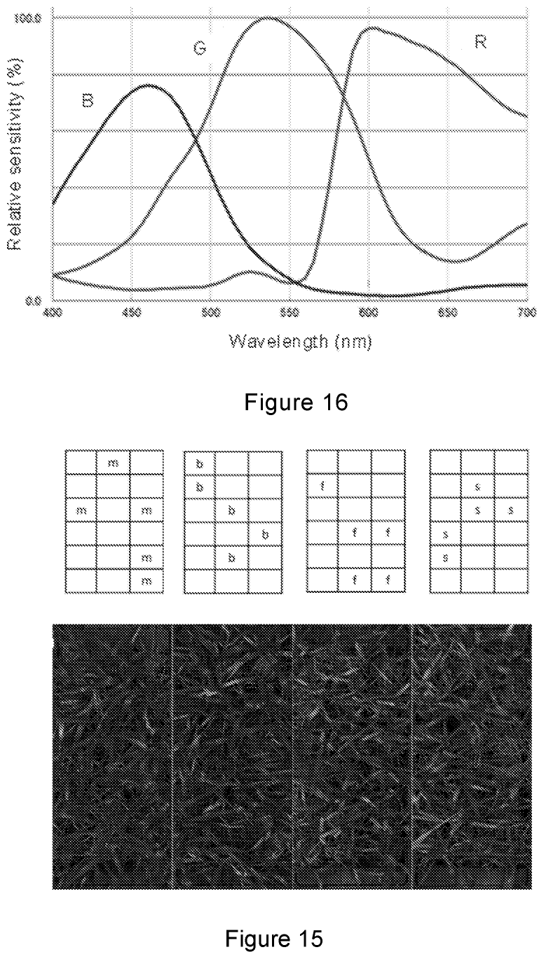

In the example shown in FIGS. 13, each array 10A of crop containers 10C represents an area of interest (AOI) with crops 10 of interest. All other areas and features around and between the arrays 10A (e.g. floor, table etc.) are of no interest for crop feature detection and should preferably be removed to reduce the noise and increase the signal for crop feature detection in step 340. As such, step 320a may further involve a further geometric correction of clipping/cropping the transformed training images so that they contain only an AOI with crops 10 of interest (whether healthy or not) and not features or objects of no interest. This step comprises determining pixel locations of the corners of the or each AOI in each transformed training images, and extracting a sub-image of the or each AOI from each transformed training image. Where there are multiple AOIs in the training images, this step may further comprise creating a composite image of the AOIs for each transformed training image of the set. In the example of FIG. 13, the corners of each array 10A are determined and the images of each array 10A are extracted, to create a composite image of the four arrays 10A for each training image of the set, as shown in FIG. 15.

In step 330, one or more field camera-specific training images are generated or extracted from each hyperspectral training image I_{TR}(x, y, λ) of the set based on the spectral response/sensitivity characteristics and spatial/pixel resolution of the field camera. The resulting field camera-specific training images have an equivalent image resolution to the images generated in the field by the field camera. Step 330 may be performed before or after steps 320a, 320b.

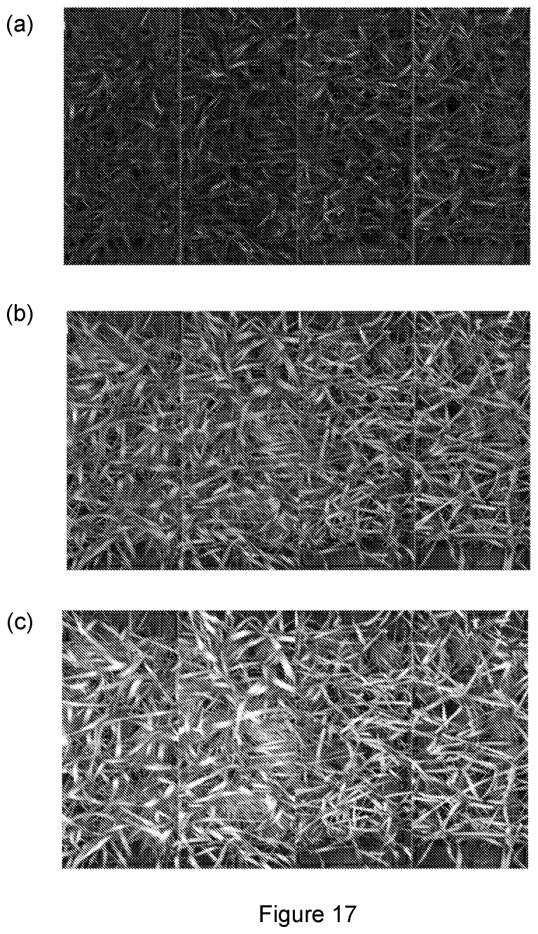

Step 330 comprises determining spectral filtering weights for the field camera at each spectral band of the training images I_{TR}(x, y, λ) based on the spectral response of the field camera and applying the filtering weights to the pixel values (i.e. intensities) of the hyperspectral training images I_{TR}(x, y, λ) at the respective spectral bands to produce one or more field camera-specific training images. In an example, the field camera-specific training images include RGB, red edge and NIR images (see FIGS. 17(a)-17(c)).

The spectral filtering weights for the field camera are determined from the spectral response/sensitivity characteristics or quantum efficiency (QE) curves of the field camera. FIG. 16 shows example QE curves for the red (R), green (G) and blue (B) spectral bands of a multi-spectral field camera. Each curve R, G and B represents the relative spectral response/sensitivity for each colour channel, which is typically associated with separate colour filters or image sensors of the field camera. The filtering weights represent the weight or coefficient needed to be applied to each spectral band of the hyperspectral training images I_{TR}(x, y, λ) in order to simulate or match the spectral response of the field camera. A different set of filter weights is determined for each spectral band of the field camera (e.g. red, green and blue in the example of FIG. 16) to simulate each spectral band of the field camera. Field-specific training images, corresponding to the types of images produced by the field camera with its spectral response, such as RGB, red edge and NIR images, can then be produced or extracted from the filtered hyperspectral training images I_{TR}(x, y, λ), as is known in the art.

FIGS. 17(a) to 17(c) show example field camera-specific RGB, NIR and red-edge images generated from the training image of FIG. 13 using the above process.

Step 330 also comprises image re-sampling (down-sampling) to decrease the spatial/pixel resolution of the training images to match that of the field camera (e.g. drone camera, mobile camera or other). Image re-sampling is based on field camera-specific equivalence parameters, such as pixel resolution, focal length, field/angle of view, aperture diameter, and/or depth of field. Equivalence parameters are known for every camera (e.g. from the technical specification of the field camera). Step 330 therefore results in one or more field camera-specific training images for each time point with the same pixel/spatial resolution and spectral characteristics as the field camera images.

Step 320a, 320b, and 330 generate field camera-specific training images in a consistent format for crop feature analysis and attribute labelling in the proceeding steps. These processing steps help to relate crop features attributes derived from training data to real world crop feature attributes derived from field camera data, and interpret and label the complex spectral and spatial image data.

In step 340, crop features and attributes are derived from the field camera-specific training images at each time instance. This step involves a similar process to that described above with reference to FIG. 8. Image segmentation is performed to identify and/or extract objects including crop features 12, 14, 16 (such as leaves, stems, heads etc.) in each image. Various image segmentation algorithms are known in the art and typically utilise edge detection (Canny or Sobel edge detection algorithms that are commonly used in the image processing field). Image segmentation defines objects in the image bounded by edges, also referred to as object polygons. Some of the detected objects are crops features, and other are of no interest. An object filter can be used to exclude objects in the image that are not crop features (e.g. soil) and/or are not of interest for crop feature classification (e.g. small objects that should be included as children of a parent object such as leaf blight). The remaining crop features 12, 14, 16 or feature polygons are then classified in terms of their geometric and spectral attributes. The geometric and spectral attributes correspond to primary attributes extracted directly from the detected crop feature polygon geometry and pixel values (see above). Geometric attributes include one or more of dimensions, area, aspect ratio, sub-feature 12a size and/or count. Spectral attributes include one or more of dominant colour, RGB, red edge and NIR pattern and hyperspectral signature, each with an associated temporal stamp and location in the image. “Patterns” refer to a collection of image features such as blobs or edges. For example, certain diseases may produce specific characteristic patterns detectable in RGB, NIR and red-edge images, such as leaf rust and stripe rust shown in FIG. 2. A hyperspectral signature refers to a spectral histogram, extracted from a specific crop feature or polygon. The spectral attributes may also include one or more remote sensing indices typically obtained through satellite images, such as NDVI and NDWI. These attributes correspond to tertiary attributes described above.

In step 350, a sub-set of classified crop features is labelled with one or more attributes by an expert based at least partly on the geometric and spectral (i.e. primary) attributes derived above in step 340 and ground control data. Ground control data may include information on the type of disease that a crop 10 has been infected with, type of pests and/or weeds present, growth conditions that the crop has been subjected to e.g. whether the crop 10 has received enough water or too much water, enough sunlight (UV) or not enough sunlight, and/or the temperature or soil (e.g. moisture) conditions. The labels may include one or more of crop feature type, healthy crop, crop with specific disease or weed or pest (e.g. septoria, blackgrass, fusarium head blight), and others. These attributes correspond to the secondary attributes described above.

Labelling may comprise labelling or tagging each crop feature with an attribute vector containing its primary, secondary and/or tertiary attributes determined or derived from the training images.

Each crop feature and its attributes can therefore be related to the same crop feature in other images taken at different points in time through the temporal stamp and location in the image. Attributes can be combined to generate unique composite or compound attributes containing richer information. For example, the observation or detection of a specific spectral signature or colour together with a specific aspect ratio may provide a stronger indicator of a certain disease or weed than any one single attribute taken in isolation. In particular, the external properties of some crops/features correlate with certain diseases. For example, blackgrass exhibits darker matter on the plant/image, which can be readily identified when combined with certain geometric properties extracted from the crop feature.

In step 360, this sub-set of labelled classified crop features is stored in the database 220 as a training data set for the machine learning model. The remaining crop features in the training images are used as test data for the machine learning model. The training data set includes at least a minimum number of classified features needed to be statistically relevant, which may be about 30%.

The machine learning model can then be trained to identify crop features in an image and determine one or more crop feature attributes for each identified crop feature using the training data set and test data, in the usual manner. Training is initially supervised learning, and can then be semi-autonomous. The result is a bespoke machine learning model trained on the most appropriate training data engineered for the specific field camera. This machine learning model can then be used in the crop monitoring method 100 describe above.

Steps 330 to 360 can be repeated for any number of field cameras, to populate the database 220.

From reading the present disclosure, other variations and modifications will be apparent to the skilled person. Such variations and modifications may involve equivalent and other features which are already known in the art, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination.

For the sake of completeness it is also stated that the term “comprising” does not exclude other elements or steps, the term “a” or “an” does not exclude a plurality, and any reference signs in the claims shall not be construed as limiting the scope of the claims.

1. A method of automated crop monitoring, comprising:
receiving image data containing a plurality of images of crops in an area of interest for monitoring;
identifying one or more crop features of each crop in each image;
determining, for each identified crop feature, one or more crop feature attributes; and
generating one or more crop monitoring outputs based, at least in part, on the crop features and crop feature attributes.

2. The method of claim 1, wherein the one or more crop monitoring outputs include one or more of: a crop feature population count, a crop feature population density map, a volumetric crop yield prediction, a crop loss map, a diseased crop map, and one or more intervention instructions.

3. The method of claim 2, wherein the one or more intervention instructions comprise instructions to apply one or more treatments to one or more regions of the area of interest, and optionally or preferably, wherein the instructions are machine integrated instructions for one or more agricultural machinery unit or vehicles to apply the one or more treatments to the one or more regions.

4. The method of claim 1, further comprising generating or updating a spatially resolved model of the identified crop features in the area of interest, wherein each crop feature is associated/tagged with an attribute vector comprising its respective one or more crop feature attributes, and optionally, wherein the model comprises a three-dimensional point cloud, where each three-dimensional point represents a crop feature associated/tagged with its attribute vector.

5. (canceled)

6. The method of claim 4, wherein the image data is generated at a first time or date, and the method comprises:
receiving second image data containing a second plurality of images of crops in the area of interest generated at a second time or date;
identifying one or more crop features of each crop in each image;
determining, for each identified crop feature, one or more crop feature attributes;
generating, based on the crop features and crop feature attributes, one or more crop monitoring outputs; and
updating the model to include the crop features and crop feature attributes for the second time or date.

7. The method of claim 1, wherein the one or more crop features in each image are identified using a machine learning model trained on a training dataset of crop images to identify the one or more crop features in the respective image based, at least in part, on one or more image features extracted from each respective image; and optionally or preferably, wherein identifying a crop feature includes identifying a crop feature type.

8. The method of claim 1, wherein determining the one or more crop features attributes comprises extracting one or more primary crop feature attributes from each identified crop feature based, at least in part, on the image pixel values and/or based on one or more image features extracted from each respective image, and wherein the one or more primary crop feature attributes include any one or more of: a location, a color, a dimension, and a sub-feature count, the location of each crop feature determined, at least in part, using geolocation data of each respective image in the image data.

9-10. (canceled)

11. The method of claim 1, wherein determining the one or more crop features attributes comprises determining one or more secondary crop feature attributes for each identified crop feature using a machine learning model trained on a training dataset of crop images to determine the one or more secondary crop feature attributes based, at least in part, on one or more image features extracted from each respective image, and wherein the one or more secondary crop feature attributes include one or more of: diseased and disease type, pest-ridden and pest type, weed-ridden and weed type, healthy, and unhealthy.

12-13. (canceled)

14. The method of claim 6, wherein the one or more image features comprise any one or more of: edges, corners, ridges, blobs, RGB colour composition, area range, shape, aspect ratio, and feature principle axis.

15. The method of claim 6, wherein the image data is generated by a field camera, and the machine learning model is trained on training data specific to the image resolution of the field camera; and optionally or preferably, wherein the training data is generated from hyperspectral images of crops in a control growth environment, and/or by the method of claim 26.

16. The method of claim 1, wherein each image is mapped to a different geolocation in the area of interest, and/or the plurality of images form an orthomosaic map of the area of interest.

17. The method of claim 1, wherein the plurality of images include multiple viewpoints of each crop in the area of interest, and the step of determining, for each identified crop feature, one or more crop feature attributes comprises:
for each identified crop feature, combining each respective crop feature attribute extracted from each respective viewpoint to provide one or more composite crop feature attributes.

18. The method of claim 1, wherein the plurality of images have a pixel resolution of at least 32 pixels per meter, and/or a pixel size of less than 25 mm.

19. (canceled)

20. The method of claim 1, comprising generating the image data using at least one field camera mounted to a drone; and
optionally mapping each image to a different geolocation in the area of interest, and/or generating an orthomosaic map of the area of interest from the plurality images.

21. A crop monitoring system, comprising:
a processing device with processing circuitry and a machine readable medium containing instructions which, when executed on the processing circuitry, cause the processing device to:
receive image data containing a plurality of images of crops in an area of interest for monitoring;
identify one or more crop features of each crop in each image;
determining, for each identified crop feature, one or more crop feature attributes;
generate one or more crop monitoring outputs based, at least in part, on the crop features a r feature attributes.

22. The system of claim 21, comprising one or more imaging systems for generating the image data, wherein the one or more imaging systems comprises a drone; and optionally wherein the drone is configured to receive flight control instructions from the processing device for generating the image data and optionally send the generated image data to the processing device.

23. (canceled)

24. The system of claim 21, comprising one or more agricultural machinery units or vehicles for applying a treatment to one or more regions of the area of interest based on the one or more intervention instructions.

25. (canceled)

26. A method of generating training data for a machine learning model used to determine crop feature attributes of crop features in images of crops generated by a field camera for crop monitoring, the method comprising:
receiving image data containing a hyperspectral training image of crops generated in a controlled growth environment using a hyperspectral training camera;
generating one or more field camera-specific training images from the hyperspectral training image, the field camera-specific training images having an equivalent image resolution to that of a field camera used to generate the field images;
identifying one or more crop features of each crop in the field camera-specific training images;
labelling a sub-set of identified crop features with the one or more crop feature attributes; and
storing the labelled classified crop features in a database as a training data set for the machine learning model.

27. The method of claim 26, wherein the step of labelling comprises determining, for each identified crop feature, one or more primary crop feature attributes based on the pixel attributes of the respective identified crop feature.

28. The method of claim 27, wherein the one or more primary attributes comprise one or more geometric and/or spectral attributes derived from the pixel attributes of the respective identified crop feature; and, optionally or preferably
wherein the geometric attributes include one or more of: location, dimensions, area, aspect ratio, sub-feature size and/or count; and/or
wherein the spectral attributes include one or more of: dominant colour, RGB, red edge and/or NIR pattern, hyperspectral signature, normalised difference vegetation index (NDVI), and normalised difference water index (NDWI).

29. The method of claim 27, wherein the step of labelling further comprises determining, for each identified crop feature, one or more secondary crop feature attributes based at least in part on the primary crop features attributes and ground control data for the crops and/or image; and, optionally or preferably wherein the ground control data comprises known information including one or more of: crop type, disease type, weed type, growth conditions, and crop age.

30. The method of claim 26, wherein the step of generating one or more field camera-specific training images comprises:
modifying the pixel values of the hyperspectral image based on the spectral response of the field camera, optionally by determining a set of spectral filter weights for each spectral band of the field camera based on the spectral response of the respective spectral band of the field camera, and applying the set of filter weights to the spectral bands of each pixel of the hyperspectral image; and
generating the one or more field camera-specific training images from the modified pixel values of the hyperspectral image; and, optionally or preferably
wherein the one or more field camera-specific training images comprise one or more of: an RGB, near infrared and red-edge image.

31. The method of claim 26, wherein the step of generating one or more field camera-specific training images comprises re-sampling the hyperspectral training image to substantially match spatial and/or pixel resolution of the field camera; and, optionally or preferably wherein the re-sampling is based on one or more equivalence parameters of the field camera.

32. The method of claim 26, wherein the image data comprises a series or plurality of hyperspectral training images of the crops, each hyperspectral training image taken at a different point in time, and wherein the method comprises:
generating one or more field camera-specific training images from each hyperspectral training image in the time series;
identifying, for each point in time, one or more crop features of each crop in the field camera-specific training images; and
labelling, for each point in time, a sub-set of identified crop features with the one or more crop feature attributes including a respective time stamp.

33. The method of claim 32, comprising applying one or more geometric and/or spectral corrections to the hyperspectral training images or the one or more field camera-specific training images associated with each different point in time to account for temporal variations in camera position and lighting conditions.

34. The method of claim 33, comprising:
assigning one of the hyperspectral training images or field camera-specific training images associated with a given point in time as a reference image;
applying a geometric transformation to the other hyperspectral training images or the other field camera-specific training images associated different points in time to substantially match the spatial location and pixel sampling of the reference image, optionally based on the location size of one or more pixels of one or more ground control points in each image; and/or
applying a white balance to the other hyperspectral training images or the other field camera-specific training images associated different points in time to substantially match the white balance of the reference image, optionally based on one or more pixels values of one or more ground control points in each image.

35. The method of claim 26, comprising training a machine learning model to identify crop features and determine crop feature attributes of crop features in images of crops generated by a field camera using the field-camera specific training images and training data set; and, optionally or preferably, wherein the machine learning model is or comprises a deep or convolutional neural network.

36. The method of claim 26, comprising generating the image data by taking a plurality of hyperspectral images over a period of time using a hyperspectral camera in substantially the same position relative to the crops; and/or
wherein each hyperspectral image is taken from substantially the same position relative to the crops.

37. (canceled)
